(19) United States
(12) Patent Application Publication
(10) Pub. No.: US 2019/0239791 A1
(43) Pub. Date: Aug. 8, 2019

(54) SYSTEM AND METHOD TO EVALUATE AND PREDICT MENTAL CONDITION

(71) Applicant: PANASONIC INTELLECTUAL PROPERTY MANAGEMENT CO., LTD., Osaka (JP)

(72) Inventors: Ariel BECK, Singapore (SG); Jovia Jia Zhen LEE, Singapore (SG); Khai Jun KEK, Batu Pahat (MY)

(21) Appl. No.: 15/888,895

(22) Filed: Feb. 5, 2018

(51) Int. Cl. A61B 5/16; G16H 50/20; A61B 5/08; A61B 3/11; A61B 5/11; A61B 5/053; A61B 5/00; A61B 5/0205

(52) U.S. Cl. CPC A61B 5/165; G16H 50/20; A61B 5/0816; A61B 5/163; A61B 3/112; A61B 5/021; A61B 5/053; A61B 5/7264; A61B 5/1123; A61B 5/4803; A61B 5/02055; A61B 5/1116

(57) ABSTRACT

The present invention relates to a system and method for monitoring and predicting the mental health of a person. Data is collected from multiple sensors including a camera and microphone. Additional sensors can be added to improve the robustness of the system such as heart rate sensors and respiration sensors. The data can be collected in phases that provide contextual awareness to the system. An algorithm can synchronize the data collected in the different phases and the data can be analyzed individually and collectively. Historical data can be included in the analysis to evaluate and predict the mental condition of a person.

### TECHNICAL FIELD

The present invention relates to a system for monitoring mental health, and more specifically, to a system and method to evaluate and predict the mental condition of a person by fusing data from different sensors collected in different contexts.

### BACKGROUND

Mental health can be defined as a level of psychological well-being or an absence of mental illness. The term mental illness refers collectively to all diagnosable mental disorders characterized by alterations in thinking, mood, or behavior associated with distress or impaired functioning. Depression is a common mental disorder that affects about 400 million globally. Other common mental disorders include dementia which affects about 35 million, and schizophrenia, which affects about 21 million people globally.

Diagnosing mental disorders can be difficult for physicians and other health care professionals. Because laboratory tests have limited value in diagnosing mental disease, a diagnosis typically relies on an evaluation by a healthcare professional. Such an evaluation typically begins with questions about symptoms and a medical history. The healthcare provider must gauge the symptoms, including any social or functional problems caused by the symptoms, and observe the patient's attitudes and behaviors. He/she must then determine if the person's symptoms and degree of disability point to a diagnosis of a specific disorder. The process is subjective and patients are prone to being misdiagnosed.

The Depression Anxiety Stress Scale (DASS-21) was developed to provide a self-report measure of anxiety, depression and stress signals. It is widely used to assess symptoms of mental suffering in adults. While the test can be helpful to diagnose mental illness, the questions are subjective in nature. Moreover, patients may not be forthcoming in accurately responding to the questions and the test may not be effective for certain demographics and mental conditions. For these reasons, there is a need for an improved method of evaluating and/or diagnosing patients for mental disorders.

Some recent efforts have focused on obtaining information and data on patients that conventionally would not be perceived and/or analyzed by a healthcare provider. For example, handwriting can be analyzed over time to detect deterioration in one's mental health. Neurological disease can affect handwriting and is common and progressive among elderly people. Information/data collected from a patient's handwriting over time can improve the reliability of a healthcare professional's diagnosis.

Physiological measures can also assist in mental health evaluations. U.S. Patent Application No. 2016/0338640 describes a psychological acute stress measurement system. Sensors measure cardiac activity of a patient, including his/her heart rate and Heart Rate Variability (HRV). HRV can be linked to psychological stress and other health ailments. While the invention can help detect and diagnose acute stress, it is not effective in diagnosing other conditions.

U.S. Patent Application No. 2009/0149778 recognizes that one's physical activity can be an important factor in evaluating him/her for depression. The invention includes a depression detection system that uses an accelerometer to measure the rest and activity of a user. However, while physical activity can be an indicator of depression, the invention does not consider other factors and/or variables in physical activity. It is also prone to error because it does not consider the context of data that is collected. Further, it cannot be used to evaluate patients for other conditions.

Accordingly, there is a need for a system that can detect and monitor a multitude of factors presented by a patient. It should be capable of analyzing and combining data to allow a healthcare professional to more accurately and objectively evaluate a patient's mental health and/or diagnose a mental condition. The system should include factors that would otherwise be unperceivable to a healthcare practitioner.

### SUMMARY OF THE INVENTION

The invention recognizes that there exists a need for a system and method to combine multiple sensors to detect and predict the mental condition of a person. The system should be capable of use to assist a healthcare provider in evaluating, monitoring and/or predicting the mental condition of a person. It should also be capable of assisting a healthcare provider in diagnosing mental health ailments.

The following summary is provided to facilitate an understanding of some of the innovative features unique to the disclosed embodiments and is not intended to be a full description. A full appreciation of the various aspects of the embodiments disclosed herein can be gained by taking into consideration the entire specification, claims, drawings, and abstract as a whole.

The invention comprises a system for evaluating and predicting the mental health of a person comprising (a) one or more sensors adapted to detect sensor data relating to the person's voluntary and autonomic responses, (b) a signal processing unit and (c) a database of historical data. Data on the person from one or more phases can be analyzed for aberrations, deviations and/or patterns in reference to historical data to evaluate the person for one or more mental health ailments. The data can also be used to predict one or more mental health ailments. The sensors can include a camera and a microphone. In an alternative design, the sensors can also include a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor and/or a heart rate sensor. A user interface can allow a healthcare provider to submit patient data from an evaluation and/or results of a Depression Anxiety Stress Scale to be included in the analysis.

The system can use computer learning and/or artificial intelligence to analyze sensor data for patterns and/or deviations correlate the patterns/deviations with mental ailments. The system can include a baseline phase, wherein the sensors identify and record baseline data on the person to establish a level from which irregularities, deviations and/or patterns are detected. Historical data can include measurements of the person that were previously detected and recorded. In the alternative, historical data can include data compiled from multiple healthy individuals and/or multiple individuals with known ailments.

The invention also includes a computer implemented method of evaluating the mental health of a person that comprises the steps of (a) detecting and recording data from the person's autonomic and voluntary responses with one or more sensors in a first phase to obtain first phase data, (b) detecting and recording data from the person's autonomic and voluntary responses with one or more sensors during a second phase to obtain second phase data and (c) comparing the first phase data with the second phase data to identify aberrations, deviations and/or patterns in the person's autonomic and voluntary responses. The aberrations, deviations and/or patterns can be indicative of the presence or absence of one or more mental health ailments.

The method can compare historical data with the first phase data and/or second phase data. The method can include the step of evaluating the mental health of the person by a healthcare provider. Data from the person can be collected during one or more additional phases. The data from each phase can be analyzed individually. It can also be analyzed collectively. The method can also include the step of predicting whether the person will experience a mental health ailment.

The sensors can include a camera and a microphone. Additional sensors can include a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor and/or a heart rate sensor.

The invention also includes a computer implemented method of evaluating the mental health of a person that comprises the steps of (a) collecting first phase data on a person's autonomic and voluntary responses with one or more sensors in a first phase, (b) detecting aberrations, deviations and/or patterns in the first phase data, (c) correlating the aberrations, deviations and/or patterns with one or more mental health ailments in historical data, (d) collecting second phase data on a person's autonomic and voluntary responses with one or more sensors in a second phase, (e) detecting aberrations, deviations and/or patterns in the second phase data by comparing the second phase data with the first phase data and (f) correlating the aberrations, deviations and/or patterns in the second phase data with one or more health ailments in historical data. The aberrations, deviations and/or patterns detected in the first phase data and the second phase data can be indicative of the presence or absence of one or more mental health ailments.

The method can include the additional steps of (g) collecting data from the person's autonomic and voluntary responses with at least one sensor during one or more additional phases, (h) detecting aberrations, deviations and/or patterns in relation to historical data and (i) correlating the aberrations, deviations and/or patterns with one or more mental health ailments found in historical data.

The sensors can include a camera and a microphone. In an alternative design, the sensors can also include a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor and/or a heart rate sensor. The step of comparing data of the person in the first phase with data of the person in the second phase and the one or more additional phases can utilize a fusion algorithm. Machine learning and/or artificial intelligence can be used to correlate the aberrations, deviations and/or patterns with one or more mental health ailments. The method can also include the additional step of predicting whether the person will experience a health ailment.

### INTRODUCTION

A first aspect of the invention is a system and method of using multiple sensors to evaluate and/or predict the mental condition of a patient based on data collected on the person in different phases.

A second aspect of the invention is a system and method of using multiple sensors to evaluate and/or predict the mental condition of a person that utilizes historical data.

A third aspect of the invention is a system and method that collects and analyzes data from a person in multiple levels/situations with contextual awareness to evaluate and/or predict the mental condition of the person.

A fourth aspect of the invention is a system and method for utilizing big data (e.g. recorded hospital records) to aid in evaluating/diagnosing a person.

A fifth aspect of the invention is a system and method for fusing data from different timelines and/or different contexts/settings to aid in evaluating/diagnosing a person.

A sixth aspect of the invention is a system and method that uses data from multiple sources and computer/machine learning to evaluate and/or predict the mental condition of a person.

A seventh aspect of the invention is a fusion algorithm that combines input from sensors (including a camera, pressure sensor and speech sensor) to evaluate and/or predict the mental condition of a person.

An eighth aspect of the invention is a system that collects physiological data on an individual and/or population under different circumstances (e.g. relaxed and stressed) to identify patterns, correlations and relationships.

A ninth aspect of the invention is an algorithm that analyzes data from handwriting sensors (e.g. writing patterns and strength/pressure of a pen) to evaluate and/or predict the mental condition of a person.

A tenth aspect of the invention is an algorithm that analyzes data from voice/speech sensors (e.g. a microphone and related software/hardware) in conjunction with other data to evaluate and/or predict the mental condition of a person based on factors such as meaning, syntax and intonation.

An eleventh aspect of the invention is an algorithm that analyzes data from sensors that detect movement/activity of a person in one or more settings to evaluate and/or predict the mental condition of the person based on factors such as fidgeting, slouching and gait.

A twelfth aspect of the invention is an algorithm that analyzes data from sensors that detect the breathing/respiration in conjunction with other data to evaluate and/or predict the mental condition of a person based on factors such as sighs and deviations from normal breathing.

A thirteenth aspect of the invention is an algorithm that synchronizes sensor input from different time periods (e.g. motion analysis data with data from handwriting analysis) to evaluate and/or predict the mental condition of a person.

A fourteenth aspect of the invention is an algorithm that fuses data from sensors (real time) with historical data to evaluate and/or predict the mental condition of a person.

A fifteenth aspect of the invention is a system that includes a database for storing data collected on an individual and/or data collected from multiple individuals (i.e. a population) to evaluate and/or predict the mental condition of a person.

### BRIEF DESCRIPTION OF THE FIGURES

The drawings described herein are for illustrative purposes only of selected embodiments and not all possible implementations, and are not intended to limit the scope of the present disclosure.

The summary above, as well as the following detailed description of illustrative embodiments, is better understood when read in conjunction with the appended drawings. For the purpose of illustrating the disclosure, exemplary constructions of the disclosure are shown in the drawings. However, the disclosure is not limited to specific methods and instrumentalities disclosed herein. Wherever possible, like elements have been indicated by identical numbers.

FIG. 1 is a flow chart the depicts the components and process for evaluating and predicting the mental condition of a person, according to one aspect of the invention.

FIG. 2 is a flow chart the details the steps for diagnosing and evaluating the mental condition of a person, according to one aspect of the invention.

FIG. 3 is a flow chart that depicts the types of data compiled for evaluating and predicting the mental condition of a person, according to one aspect of the invention.

FIG. 4 is a flow chart that depicts the independent and collective analysis of data from multiple phases, according to one aspect of the invention.

### DETAILED DESCRIPTION OF THE INVENTION

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “aberration” refers to a departure from what is normally observed or expected. Similarly, a “deviation” refers to a departure from an established course or accepted standard and can be determined statistically. A “pattern” refers to a regular and intelligible form or sequence of acts, tendencies or other observable characteristics discernible in the way in which something occurs.

The term “algorithm” refers to a procedure for solving a mathematical problem in a finite number of steps that frequently involves repetition of an operation.

The term “artificial intelligence” or “AI” refers to intelligence exhibited by machines, rather than humans. The term, as applied herein, refers to when a machine mimics “cognitive” functions that humans associate with other human minds, such as “learning” and “problem solving.”

The term “autonomic” refers to actions that are made unconsciously. Most autonomic actions are involved in regulating bodily functions such as the heart rate, digestion, respiratory rate and pupillary response. This system is the primary mechanism in control of the fight-or-flight response. In contrast, “voluntary” actions are those that a person consciously makes.

The term “big data” refers to large data sets that can be analyzed computationally to reveal patterns, trends, and associations, especially relating to human physiological responses, human behavior and interactions. This can include, for example, a databases of electronic health records from a hospital or clinic.

The term “classifier” refers to the mathematical function, implemented by a classification algorithm that maps input data to a category. An algorithm that implements classification, especially in a concrete implementation, is known as a classifier.

The term “computer learning” or “machine learning” refers to an application of artificial intelligence (AI) that provides systems the ability to automatically learn and improve from experience without being explicitly programmed. Machine learning focuses on the development of computer programs that can access data and use it learn for themselves.

The term “cognitive” refers to of, relating to, being, or involving conscious intellectual activity (as thinking, reasoning, or remembering).

The term “Controller Area Network,” “CAN” or “CAN bus” refers to a vehicle bus standard designed to allow microcontrollers and devices to communicate with each other in applications without a host computer.

The term “data fusion system” refers to a system that can align/integrate data sets and combine them to produce a meaningful result or conclusion.

The term “Depression Anxiety Stress Scale” or “DASS” refers to a test made up of 42 self-report items, each reflecting a negative emotional symptom. Each of these is rated on a four-point Likert scale of frequency or severity of a person's experiences over the last week with the intention of emphasising states over traits.

The term “fusion” or “data fusion” refers to the process of integration of multiple data and knowledge representing the same real-world object into a consistent, accurate, and useful representation.

The term “healthcare provider,” “healthcare practitioner,” “doctor,” “physician,” “psychologist” or “psychiatrist” refers to an individual involved with the testing, analysis and/or treatment using the system and/or methods described herein.

The term “heart rate variability” or “HRV” refers to the a measure of the beat-to-beat variations in heart rate. It is related to the regulation of the heart by the autonomic nervous system. Several conditions can also be associated with modified (usually lower) HRV, including diabetic neuropathy and depression.

The term “historical data” refers to data (e.g. physiological measurements and actions of individuals) recorded and/or stored in a database that can be accessed for analysis and/or comparison. The data can be raw (e.g. sensor data) or processed (e.g. fused data). Historical data can include (a) data compiled from groups/populations of individuals and (b) data compiled from an individual person. For example, data can be recorded from healthy people and people with known ailments. An analysis of the data can indicate variations in physiological measurements that can be correlated with ailments. Likewise, data from an individual person can be recorded and stored. This can allow the system to identify patterns, variations and/or aberrations in activity for that particular person.

The term “impairment” refers to any number of conditions that may reduce or negatively affect a person's ability to function, including drowsiness, fatigue, distraction, intoxication, illness, anxiety or agitation.

The term “micro-expression” refers to a brief, involuntary facial expression on the face of humans according to emotions experienced. Micro-expressions occur when a person is consciously trying to conceal all signs of how they are feeling, or when he/she does not consciously know how they are feeling. Unlike regular facial expressions, it is difficult/impossible to hide micro-expression reactions. Micro-expressions cannot be controlled as they happen in a fraction of a second, but it is possible to capture them with a high speed camera.

The term “mental illness” or “mental health ailment” refers to a condition that causes serious disorder to a person's behavior and thinking. While there are a myriad of mental illnesses, common illnesses include stress, anxiety, depression, dementia and schizophrenia.

The term “module” refers to a self-contained unit, such as an assembly of electronic components and associated wiring or a segment of computer software, which itself performs a defined task and can be linked with other such units to form a larger system.

The term “phase” refers to a distinct period of time during which the invention observes a person in a known environment and context. Context includes, but is not limited to, location, people in the venue, setting (time, day, date etc.). For example, a first phase can be the duration when a subject enters a first location (e.g., the waiting room of a clinic) and is monitored by sensors. A second phase can be the duration when a subject enters a second location (e.g., a meeting room and interview by a doctor) and is monitored by sensors. The location of first and second phase can be the same or different. Likewise, the duration for first phase and second phase can be the same or different.

The term “pupillometry” refers to the measurement of pupil diameter. Pupillary responses can reflect activation of the brain allocated to cognitive tasks. For example, greater pupil dilation can be associated with increased processing in the brain.

The term “signal processing unit” refers to a computer component that can analyze, synthesize, and modify signals, which are broadly defined as functions conveying, “information about the behavior or attributes of some phenomenon”, such as sound, images or biological measurements.

The term “symptom” refers to subjective evidence of disease, while a “sign” refers to objective evidence of disease. Thus, a symptom is a phenomenon that is experienced by the individual affected by the disease. A sign is a phenomenon that can be detected by someone other than the individual affected by the disease

Other technical terms used herein have their ordinary meaning in the art that they are used, as exemplified by a variety of technical dictionaries.

### DESCRIPTION OF PREFERRED EMBODIMENTS

The particular values and configurations discussed in these non-limiting examples can be varied and are cited merely to illustrate at least one embodiment and are not intended to limit the scope thereof.

While the invention is primarily described for monitoring and predicting a person's mental condition, it is understood that the invention is not so limited and can be used to assist with other endeavors that require monitoring a person's mental health and/or diagnosing a person for mental disorders. The invention can be used in long term care environments (e.g. convalescent homes) to monitor patient health and/or monitor patient reaction to medications. It can also be used to monitor the health and detect deterioration of mental health over time in people who experience stressful situations (e.g. law enforcement, emergency services, military, etc.).

The invention can also be used outside of the clinical environment. Other applications include, for example, using the invention in evaluating employees such as teachers to screen for mental health. The invention can also be used to screen airplane pilots, truck drivers and heavy equipment operators. Further, the invention can be used to monitor emotions (e.g. stress and anxiety) of a person. In this context, the invention can be used to monitor the levels of satisfaction of customers when, for example, providing customer feedback.

The invention recognizes that a person/patient can exhibit different physical and/or physiological responses in different environments and as a result of different stresses. There is a need for a system and method to combine multiple sensors to detect actions and physiologic activities of a person. Contextual awareness and patterns of activity should be considered. The responses can be used to evaluate and predict the mental health of an individual.

The invention can include sensors to detect parameters that are not perceivable using conventional methods of mental health evaluations. Sensors and Artificial Intelligence (AI) can be used to evaluate mental health, diagnose mental conditions and/or assist healthcare practitioners in diagnosing medical conditions.

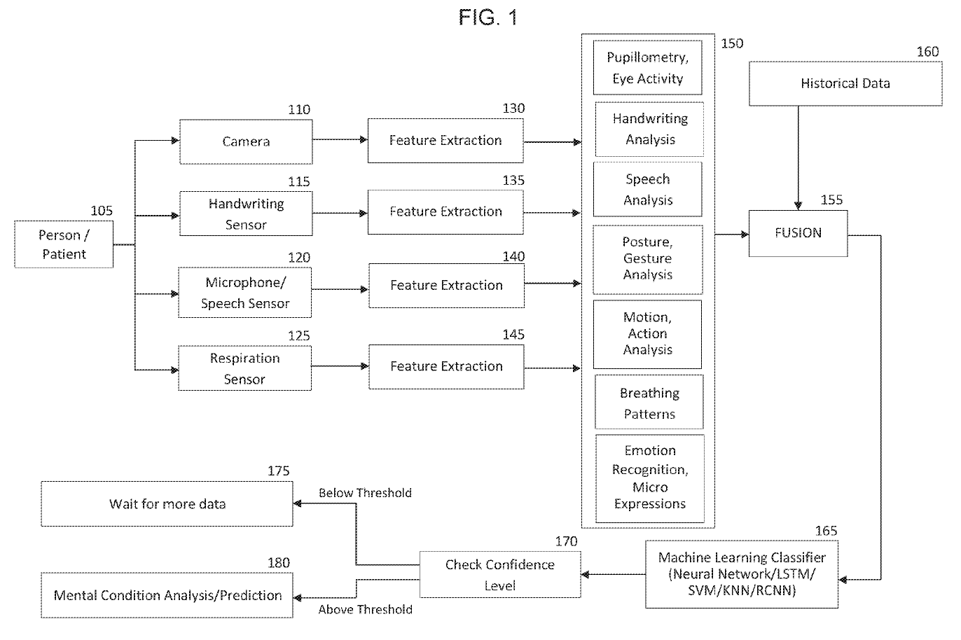

FIG. 1 is a schematic diagram of a system for evaluating and predicting the mental health of a person, according to one aspect of the invention. A person 105 can be monitored by a plurality of sensors in different environments under different conditions. The sensors can be real time, continuous (e.g. motion sensors) as well as real time, non-continuous (e.g. sound activated microphone). Both autonomic and voluntary responses of the person can be recorded and analyzed.

At a minimum, the system can include a camera 110 and a microphone 120. The camera can detect stress, anxiety or other emotions from expressions, micro-expressions and/or spontaneous expressions. The microphone can detect the same emotion from the person's voice. However, as depicted, the sensors include a camera 110, a handwriting sensor 115, a microphone/speech sensor 120 and a respiration sensor 125. And although four sensors are illustrated, the system can include an array of various sensors for redundancy and improved robustness.

For example, the system can include eye sensors, pupillometry sensors, face analysis sensors, body position sensors, body posture sensors, audio sensors, accelerometers, motion sensors, heart rate sensors and other physiologic sensors. The sensors can monitor physiological activity of the patient including: heart/pulse rate, blood pressure, respiration rate/pattern, heart rate variability, body posture, body movement, electromyographic data, head position, head movement, eye direction, eye movement, gaze pattern/direction, eyelid opening, blink rate, eyebrow activity, pupil size, pupil activity, facial expressions, facial activity, speaking activity, perspiration, EEG (electroencephalography), ECG (electrocardiography), EMG (electromyography), skin conductance, changes in speaking tone and/or speaking volume. One or more ambient light sensors can be used to account for external lighting that can affect the patient's pupils. The system can also include monitors to detect substances secreted from the patient (e.g. metabolites) as well as substances in the patient's system such as alcohol, drugs and/or drug metabolites.

### Camera

The system can include one or more cameras 110 and incorporate data related to a patient's posture and motions. The camera(s) can also detect and monitor facial expressions, including micro-expressions. Further, eye movement and pupillometry can also be recorded/analyzed. More specifically, the camera(s) can be detect the following:

1) Micro-expressions and facial gestures;

2) Slouching and/or irregular or impaired posture;

3) Irregular and/or erratic movements;

4) Slow or delayed reaction times; and

5) Facial behavior (e.g. expressions and movement).

Aberrations of baseline behavior can be indicative of, for example, a high level of anxiety.

In a preferred design, the system uses one or more cameras 110 to monitor the body posture/movement, facial expressions and eyes/pupils. By observing one's body, the system can detect and monitor behavior and regular movement as well as erratic or unnatural movements. For example, stumbling, stupor and/or diminished posture can be a factor in evaluating a patient for mental health.

The camera(s) can also monitor one's head direction, posture and facial expressions. The amount of eye contact that the patient makes with a healthcare provider can be monitored and considered. This is analyzed by considering the direction of the patient's head and body position as well as his/her eye direction, focus and glance pattern. Increased cognitive load can be demonstrated by:

1) Longer fixations;

2) Gazing toward the center of the field of view; and

3) Frequent/sporadic glances away from a healthcare provider.

Pupil Diameter can indicate anxiety and/or cognitive distraction. The system can account for the patient's typical pupil responses as well as individual differences.

### Handwriting Sensors

The system can also include handwriting sensors 115. The pressure exerted by a patient when writing can have clinical significance. For this reason, pressure sensors can be included on a writing instrument. Further, a computer vision algorithm can be included to analyze handwriting (e.g. pattern, pressure exerted on pen). The response time for answering individual questions can also be observed and recorded. Similarly, the response time for completing the entire set of questions can also be observed and recorded. Further, handwriting can be analyzed over time to detect deterioration.

### Microphone/Speech Sensors

The system can also include a microphone and/or speech sensors 120. Deviations from normal speech patterns can be identified. A patient's speech can be analyzed over time to detect aberrations and possible signs of anxiety and/or declining mental health.

### Respiration Sensor

The system can also include a breathing/respiration sensor 125. The sensor can be, for example, a belt respirator, girth respirator or microphone/audio respirator. Aberrations and possible signs of anxiety or other condition can be correlated with changes in respiration.

Data (i.e. raw data) from the sensors can be extracted (130, 135, 140 and 145) to a module or central computer 150. An algorithm can then be used to analyze the features/data compiled from the sensors. Machine learning and/or artificial intelligence (not shown) can be used to detect aberrations, deviations and/or patterns. A fusion step 155 can combine the data into a usable form and/or numerical format that can be used for further analysis. One or more algorithms can be used to weigh/adjust the value of data obtained from each sensor. This step can incorporate historical data 160.

### Historical Data

Historical data 160 can include physiological data stored in a database. This allows data to be used in future evaluations of the individual. For example, the system can identify physiological parameters of the individual during relaxed settings (e.g. speaking to a friend or family member outside of a clinical setting). Physiological data can also be collected when the individual speaks to a healthcare provider in a clinical setting. The system can identify patterns, variances as well as aberrations from baseline/benchmark levels.

Historical data can also include physiological measurements from individuals with known mental health diseases and conditions. For example, the system can use data from patients suffering from an ailment (e.g. depression, anxiety, PTSD, etc.) for comparison to those of healthy individuals as well as the data of a particular patient.

The system can identify physiological variations based on compiling clinical data. Historical data can be compiled by, for example, data mining. An algorithm and/or machine learning can be used to identify patterns and/or characteristics that are common with ailments in mental health. For example, machine learning can be used to analyze a group of patients. The system can identify physiological measurements that are common to individuals that are affected with an ailment or that have become affected with an ailment over a period of time.

The system allows vast amounts of data to be collected in a database. Machine Learning 165 and/or artificial intelligence can be utilized to, for example, identify patterns, relationships and/or correlations among the data. The system can also predict the mental condition of a patient. For example, the system can recognize a decline in cognitive abilities. The system can also identify patterns of conduct/activity of patients in historical data that have subsequently experienced mental health issues. In this situation, it can predict a mental health condition before it would otherwise be diagnosable.

The end result is an analysis/diagnosis of the mental condition of a person or patient. The system can use a confidence factor based on the amount of data collected/utilized as well as the deviations from normal/predicted levels. For example, a person with multiple factors that indicate a high likelihood of a mental condition would have a high confidence level (e.g. 90% likelihood). The system would give a low confidence level (e.g. 50% likelihood) in a situation with limited data and no strong indicators. A high confidence level can indicate a mental condition and/or a prediction of a mental condition 180. Likewise, a low confidence level can indicate that more data is necessary for an evaluation 175.

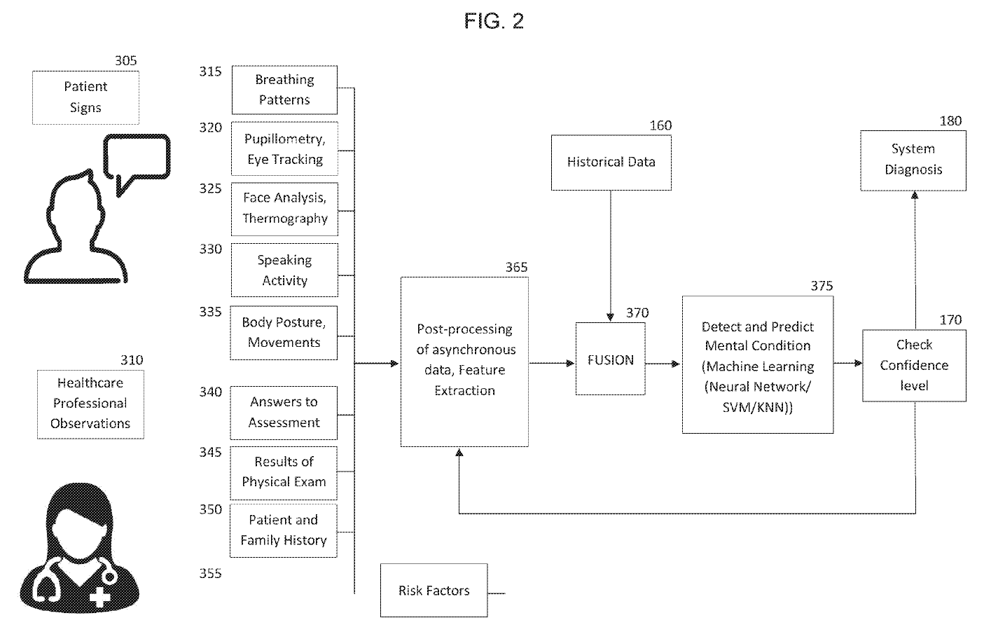

FIG. 2 is a schematic diagram that depicts some signs and symptoms that can be monitored and recorded by the system. In a preferred embodiment, the invention includes sensors that detect patient signs 305. Sensors can include those described/illustrated in FIG. 1. The patient signs are primarily objective in nature. These can include breathing patterns 315 pupillometry/eye tracking 320, face analysis and thermography 325, speaking activity 330 and body posture/movements 335.

### Breathing Patterns

The system can detect and record respiration rates. Breathing patterns can include sighs, deep breathing and aberrations from normal breathing patterns. This, along with other data, can assist in detecting and/or predicting mental health conditions.

### Pupillometry

Pupil size can vary with mental health. Dilated pupils can occur with anxiety, and is most common during periods of intense anxiety that occur conditions such as panic disorder/panic attacks, PTSD and phobias.

Facial Expression and/or Micro-Expression

Facial expressions can be indicative of anxiety. Abnormal facial expressions can also occur as a result of conditions that damage the nerves to the face, such as Bell's palsy or facial paralysis. Damage to the brain, such as with stroke or transient ischemic attack, can also cause impaired movement, including changes in facial expressions. Patients with certain psychiatric conditions can have abnormal facial expressions, particularly the psychotic disorders, in which an one's sense of reality is impaired. Facial tics, such as those that occur in Tourette syndrome, are one form of abnormal facial expressions. The system can monitor head direction, his/her posture and facial expressions. Eye direction and head position can also be monitored.

### Facial Temperature

Recent studies suggest that facial temperature can be affected by mental health disorders. Thermal infrared imaging cameras can be included to monitor a patient's facial temperature. The temperature of regions of the face can be compared to previously measured temperatures of the patient. The readings can also be compared with average readings. Skin gas can similarly be analyzed.

### Speech/Voice

One's speaking voice and cadence can reflect his/her mental health. The speech of the patient can be compared to previously recorded/analyzed speech of the patient. Data from the speech can also be compared with data obtained from healthy and/or patients with a known mental illnesses. Speech can be analyzed for meaning, syntax and intonation.

### Body Posture and Gesture/Action Analysis

Sensors can detect (often subtle) actions such as gestures, fidgeting and/or slouching. Such actions can be indicative of, for example, a person's attitudes and level of comfort/anxiety.

Data can also be collected from the observations/analysis of a healthcare provider 310. This data is often subjective in nature, including answers to an assessment 340, results of a physical examination 345, patient and family history 350, and risk factors 355 such as one's work environment. This data can be entered through an interface and then assigned numerical value(s) for incorporation into the system's evaluation.

Data from the sensors can undergo post-processing of synchronous data, feature extraction 365 and fusion 370. Historical data 160 can be included in the fusion process. By fusing data from multiple sensors, the system can detect and predict mental conditions 375.

The system can detect aberrations, deviations and/or patterns in the data collected from the sensors. Both raw data and processed data can be stored and analyzed. The aberrations, deviations and/or patterns can be correlated with aberrations, deviations and/or patterns that are attributable to one or more mental health ailments. This step 375 can utilize big data and machine learning. For example, the system can detect a change in posture as a result of increased muscle tension. It can also detect changes in speaking patterns (e.g. more erratic speaking). These can be indicative of aberrations/deviations from one's normal behavior and be correlated/associated with anxiety. The system can use historical data to correlate these signs/symptoms with similar signs/symptoms from patients known to have anxiety.

Patterns of activity can also be analyzed. For example, an isolated instance of changed posture could be attributed to muscle soreness. The posture of the patient would improve as the muscle soreness dissipates. However, if the posture and erratic speaking become more pronounced, the system can acknowledge the likelihood that the patient is suffering from anxiety. Further, the system can predict that the patient will become depressed based on the pattern.

The aberrations, deviations and/or patterns can be compared to one or more baseline values. The baseline values can be derived from the subject and/or from the general population. In the alternative, baseline values can be established using data from a first phase. If a change is detected in a second (or other) phase beyond a certain threshold, it can raise an alarm and indicate the possibility of one or more mental health ailments.

The system can check the confidence level at this stage 170. A high confidence level can indicate a mental condition (i.e. diagnosis) and/or a prediction of a mental condition 180. A low confidence level can indicate that more data is necessary. Data collected from multiple settings/contexts will typically lead to a higher confidence level. Input from a healthcare provider (e.g. from an evaluation and/or results of a DASS-21) can further increase the confidence level.

### WORKING EXAMPLE

### Evaluation of Mental Health of a Patient

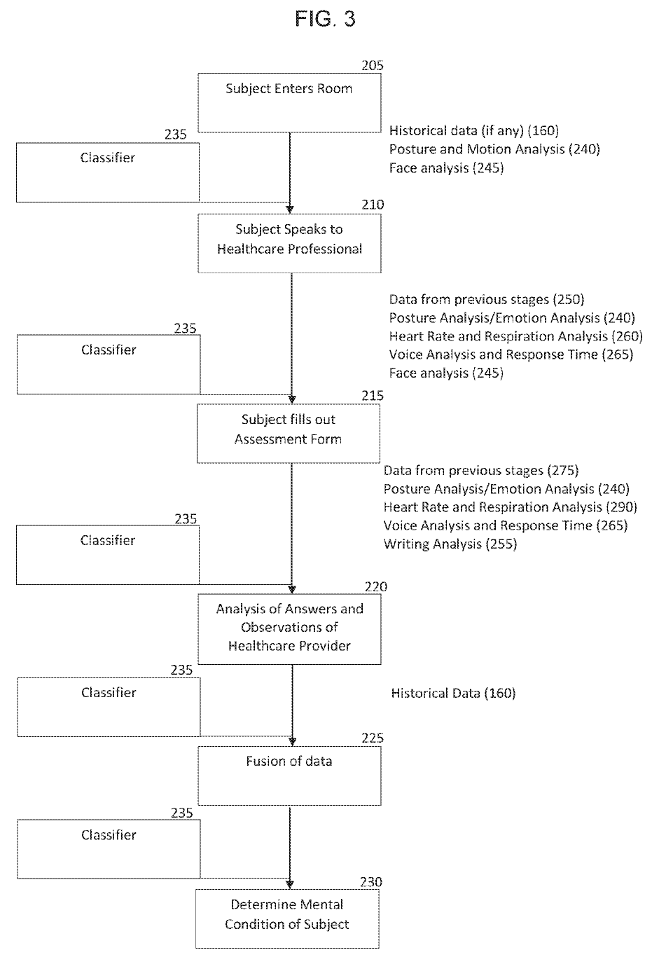

The invention recognizes that a patient can exhibit different signs/symptoms depending on his/her environment and/or situation. FIG. 3 depicts one aspect of the invention, wherein a subject/patient is monitored in different environments under different circumstances.

The subject can exhibit signs of stress and anxiety when he/she enters a clinic or hospital setting. The same subject can exhibit signs of depression when he/she is at home alone. The system and method can collect and analyze data from a subject in multiple situations or phases (e.g. clinical and non-clinical). In this example, the subject enters a clinic and is evaluated for mental health. Upon entering the clinic, data is collected from multiple sensors. The system continues to monitor the subject as he/she speaks with a healthcare provider and completes the questionnaire for a Depression Anxiety Stress Scale (DASS-21). Data is collected from the subject and his/her actions that would otherwise not be perceived or reported to a healthcare provider.

Context (i.e. the interrelated conditions in which actions are observed) can be important to evaluating/analyzing the data. In this example, the context of the data will be apparent based on the fixed location of the sensors. Sensors in a waiting room can collect data in a first context or “phase.” Sensors in an examining room can collect data in a second phase.

In an alternative embodiment, the sensors can be mobile and/or wearable. For example, a mobile camera and/or microphone can accompany the patient into different settings. In this regard, one or more smartphones can be used for sensors. Context can be entered through a user interface. In the alternative, the system can identify context based on patterns of activity/conduct expressed by the patient. For example, the system can identify speech and breathing patterns that are associated with a patient speaking to a healthcare provider 210. Sensors can identify movements associated with sitting and writing when the subject completes an assessment form 215.

I. Establishment of Baseline Habits and Characteristics

The system can detect and analyze a subject's normal habits and characteristics to establish baseline levels of activity. For example, the subject can be monitored as he/she enters a room or office 205. The system can utilize factors such as historical data 160, his/her posture and motion 240 as well as facial activity/expressions 245. At each phase, a classifier or classification algorithm 235 identifies the proper category for the data collected.

In the alternative, a patient can be monitored during his/her daily routine to detect and record normal actions and patterns of actions. This information can be saved/recorded on the system or remotely, for example, on cloud storage. The baseline characteristics can thereafter be used to detect irregularities and/or deviations. For example, the system can detect normal respiration activity and variations for the particular patient. This allows the system to identify irregular breathing, such as an increased rate, deeper breathing and/or sighs. Baseline levels of other actions (e.g. body posture/movements, face analysis, facial thermography, speaking activity and pupillometry/eye activity) can be recorded and analyzed in the same manner. Characteristics of the actions and/or patterns of the actions can be identified and analyzed. The data can assist in detecting and/or predicting a mental health condition by comparison to data in a clinical situation/setting.

II. Patient Monitoring During Consultation with Provider

The system can monitor a patient during interaction with a healthcare provider 210. The patient can display different signs/symptoms from baseline levels because of the different context. The system can evaluate the patient by utilizing factors such as data from the previous stage 250, his/her posture and motion 240, heart rate and respiration data 260, voice analysis 265 as well as facial activity/expressions 245. At each stage, the confidence level increases as more data is available to the system.

III. Patient Monitoring while Completing Forms

The system can monitor the patient as he/she completes paperwork 215. For example, he/she may complete a Depression Anxiety Stress Scale (DASS-21). The system can utilize factors such as data from the previous stage 275, his/her posture and motion 240, heart rate and respiration data 260, voice analysis and response time 265, as well as writing analysis 255.

IV. Analysis of Answers and Observations of Provider

The system can analyze the patient's answers to the written assessment (e.g. DASS-21) for indications of mental health ailments. A healthcare provider can analyze the answers to the assessment along with his/her observations of the subject 220. The healthcare provider can enter his/her observations and comments into a user interface. The system can utilize historical data 160 in the process of evaluating the patent.

V. Fusion of Data

Historical data 160 can be included in an evaluation/diagnosis. The data can be fused 225 to determine the mental condition of the subject. At this step, data submitted by a healthcare provider can be assigned one or more numerical values so that it is incorporated into the computer implemented method. A confidence level (not shown) can thereafter determine whether the system should continue collecting data for a more reliable determination.

In a preferred method, the system does not use controlled stimuli so that a person can be observed during his/her natural interaction. The sensors can be integrated within the environment without requiring the subject to perform any particular action. This allows a subject to be observed person during his/her normal habits and interactions. The system can continuously observe/monitor a subject without being prompted to take any actions.

Accordingly, it is possible to observe a person without informing him/her of the system and/or sensors. Thus, observations are made while a person is in his/her natural state. It is also possible to inform a person of the system in order to obtain consent. Thereafter, they can be encouraged to disregard sensors and act naturally. Because the subject is not prompted to take particular action, the system can observe natural and uncontrolled behavior in a “semi-controlled” environment.

Although the FIG. 3 depicts a single person or patient being monitored, it is possible to monitor multiple patients. The system can include additional features to identify a particular person (e.g. facial recognition technology). Data collected for an individual can be appropriately identified and stored. Accordingly, the system can continue to collect data as multiple patients enter a room or clinic from day to day.

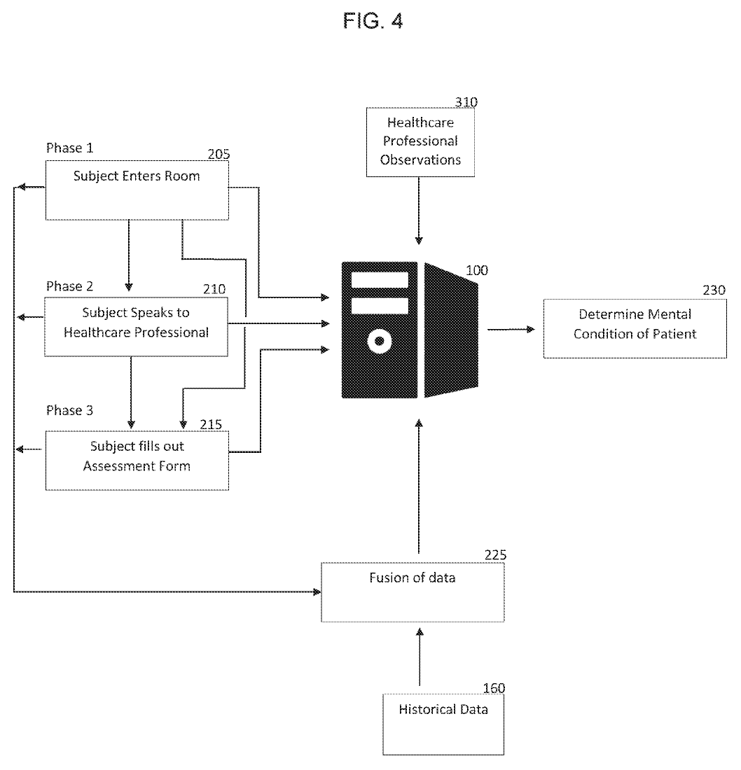

FIG. 4 depicts input into the system from different phases. Data can be collected from different phases (i.e. setting and time periods) to analyze a subject. The first phase is depicted as the period when the subject enters the room/office 205. The second phase is depicted as the period when the subject speaks to a healthcare professional 210. The third phase is depicted as the period when the subject fills out an assessment form 215. Although three phases are illustrated, the system can include additional phases. For example, a subject can be observed outside of the clinic while he/she interacts with someone other than a healthcare professional (not shown). Portable sensors with wireless connectivity can be placed in different settings for this purpose.

Different situations or contexts can affect a person's emotion. The system can recognize differences in data between phases that can be significant. For example, a person's heart rate and breathing patterns (physiological signs) can change from the first phase to the second phase despite showing no changes in his/her physical behaviors or responses. This could be the result of reluctance to speak or inform about one's health problems. A more pronounced change could indicate that the person suffers from anxiety or another mental health ailment.

The data from each phase can be analyzed independently and collectively. In each phase, the system can identify variations and/or aberrations in activity that can be indicative of one's mental health. In the first phase 205, data is analyzed and recorded in the system 100. This data can also be utilized for analysis in the second phase 210. Likewise, data from both the second phase 210 can be independently analyzed and recorded in the system 100. Data from the first phase 205 and second phase 210 can be considered together (i.e. collectively).

Data from the third phase 215 can also be independently analyzed and recorded in the system 100. Data from both the first, second and third phase can be utilized (collectively) for analysis. As additional data is available, the confidence level should be higher.

The data from each phase can also be fused 225 and analyzed with historical data 160. A user interface can allow a healthcare professional 310 to submit data into the system for consideration. This can include, for example, observations from contact with the patient (including a diagnosis), results from a DASS-21 (or equivalent) as well as data that is not dependent on time such as handwriting analysis.

Input from a healthcare provider can serve multiple purposes. It can provide feedback to the system in regard to accuracy. For example, a healthcare professional can diagnose and acknowledge that a patient is suffering from depression. Positive feedback will result if the system reached the same conclusion independently. The healthcare provider's input can also provide historical data. After a diagnosis by a healthcare professional, the system can categorize the particular patient with his/her known ailment. This diagnosis can be incorporated into the system's historical data.

Operating Environment:

The system is typically comprised of a central server that is connected by a data network to a user's (e.g. healthcare provider's) computer. The central server can be comprised of one or more computers connected to one or more mass storage devices. The precise architecture of the central server does not limit the claimed invention. Further, the user's (i.e. healthcare practitioner's) computer can be a laptop or desktop type of personal computer. It can also be a cell phone, smart phone or other handheld device, including a tablet. The precise form factor of the user's computer does not limit the claimed invention. Examples of well-known computing systems, environments, and/or configurations that may be suitable for use with the invention include, but are not limited to, personal computers, server computers, hand held, laptop or mobile computer or communications devices such as cell phones and PDAs, multiprocessor systems, microprocessor-based systems, set top boxes, programmable consumer electronics, network PCs, minicomputers, mainframe computers, distributed computing environments that include any of the above systems or devices, and the like. The precise form factor of the user's computer does not limit the claimed invention. In one embodiment, the user's computer is omitted, and instead a separate computing functionality provided that works with the central server. In this case, a user would log into the server from another computer and access the system through a user environment.

The user environment can be housed in the central server or operatively connected to it. Further, the user can receive from and transmit data to the central server by means of the Internet, whereby the user accesses an account using an Internet web-browser and browser displays an interactive web page operatively connected to the central server. The central server transmits and receives data in response to data and commands transmitted from the browser in response to the customer's actuation of the browser user interface. Some steps of the invention may be performed on the user's computer and interim results transmitted to a server. These interim results may be processed at the server and final results passed back to the user.

The methods described herein can be executed on a computer system, generally comprised of a central processing unit (CPU) that is operatively connected to a memory device, data input and output circuitry (I/O) and computer data network communication circuitry. Computer code executed by the CPU can take data received by the data communication circuitry and store it in the memory device. In addition, the CPU can take data from the I/O circuitry and store it in the memory device. Further, the CPU can take data from a memory device and output it through the I/O circuitry or the data communication circuitry. The data stored in memory may be further recalled from the memory device, further processed or modified by the CPU in the manner described herein and restored in the same memory device or a different memory device operatively connected to the CPU including by means of the data network circuitry. The memory device can be any kind of data storage circuit or magnetic storage or optical device, including a hard disk, optical disk or solid state memory. The I/O devices can include a display screen, loudspeakers, microphone and a movable mouse that indicate to the computer the relative location of a cursor position on the display and one or more buttons that can be actuated to indicate a command.

The computer can display on the display screen operatively connected to the I/O circuitry the appearance of a user interface. Various shapes, text and other graphical forms are displayed on the screen as a result of the computer generating data that causes the pixels comprising the display screen customer's actuation of the browser user interface. Some steps of the invention can be performed on the user's computer and interim results transmitted to a server. These interim results can be processed at the server and final results passed back to the user.

The invention may also be entirely executed on one or more servers. A server may be a computer comprised of a central processing unit with a mass storage device and a network connection. In addition a server can include multiple of such computers connected together with a data network or other data transfer connection, or, multiple computers on a network with network accessed storage, in a manner that provides such functionality as a group. Practitioners of ordinary skill will recognize that functions that are accomplished on one server may be partitioned and accomplished on multiple servers that are operatively connected by a computer network by means of appropriate inter process communication. In addition, the access of the website can be by means of an Internet browser accessing a secure or public page or by means of a client program running on a local computer that is connected over a computer network to the server. A data message and data upload or download can be delivered over the Internet using typical protocols, including TCP/IP, HTTP, TCP, UDP, SMTP, RPC, FTP or other kinds of data communication protocols that permit processes running on two remote computers to exchange information by means of digital network communication. As a result a data message can be a data packet transmitted from or received by a computer containing a destination network address, a destination process or application identifier, and data values that can be parsed at the destination computer located at the destination network address by the destination application in order that the relevant data values are extracted and used by the destination application. The precise architecture of the central server does not limit the claimed invention. In addition, the data network may operate with several levels, such that the user's computer is connected through a fire wall to one server, which routes communications to another server that executes the disclosed methods.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as C, C-HF, C#, Action Script, PHP, EcmaScript, JavaScript, JAVA, or 5 HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The invention may be described in the general context of computer-executable instructions, such as program modules, being executed by a computer. Generally, program modules include routines, programs, objects, components, data structures, etc., that perform particular tasks or implement particular abstract data types. The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card), or other memory device. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated but connected by a data network in a manner so that they operate together to execute the process steps. In one embodiment, a user's computer can run an application that causes the user's computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In one embodiment, the relational database may be housed in one or more operatively connected servers operatively connected to computer memory, for example, disk drives. In yet another embodiment, the initialization of the relational database may be prepared on the set of servers and the interaction with the user's computer occur at a different place in the overall process.

It should be noted that the flow diagrams are used herein to demonstrate various aspects of the invention, and should not be construed to limit the invention to any particular logic flow or logic implementation. The described logic may be partitioned into different logic blocks (e.g., programs, modules, functions, or subroutines) without changing the overall results or otherwise departing from the true scope of the invention. Oftentimes, logic elements may be added, modified, omitted, performed in a different order, or implemented using different logic constructs (e.g., logic gates, looping primitives, conditional logic, and other logic constructs) without changing the overall results or otherwise departing from the true scope of the invention.

It will be appreciated that variations of the above disclosed and other features and functions, or alternatives thereof, may be combined into other systems or applications. Also, various unforeseen or unanticipated alternatives, modifications, variations or improvements therein may be subsequently made by those skilled in the art which are also intended to be encompassed by the following claims.

Although embodiments of the current disclosure have been described comprehensively, in considerable detail to cover the possible aspects, those skilled in the art would recognize that other versions of the disclosure are also possible.

What is claimed is:

1. A system for evaluating and predicting the mental health of a person comprising:
a. one or more sensors adapted to detect sensor data relating to the person's voluntary and autonomic responses;
b. a signal processing unit; and
c. a database of historical data,
wherein sensor data is detected and recorded in at least one phase; and
wherein sensor data from the at least one phase is analyzed for aberrations, deviations and/or patterns in reference to historical data to evaluate the person's mental health and predict one or more mental health ailments.

2. The system of claim 1, wherein the one or more sensors comprise a camera and a microphone.

3. The system of claim 1, wherein the one or more sensors comprise a camera, a microphone and at least one of a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor and a heart rate sensor.

4. The system of claim 1, wherein the system uses computer learning and/or artificial intelligence to analyze sensor data for aberrations, deviations and/or patterns in reference to historical data to evaluate the person for one or more mental health ailments.

5. The system of claim 1, wherein the system further comprises a user interface for a healthcare provider to submit patient data from a patient evaluation and/or results of a Depression Anxiety Stress Scale, and
wherein patient data is included to evaluate the person for one or more mental health ailments.

6. The system of claim 1, wherein the at least one phase comprises a baseline phase,
wherein the sensors identify and record baseline sensor data on the person to establish a level from which aberrations, deviations and/or patterns are detected.

7. The system of claim 1, wherein historical data comprises sensor data of the person that was previously detected and recorded.

8. The system of claim 1, wherein historical data comprises sensor data compiled from multiple healthy individuals and/or multiple individuals with known mental health ailments.

9. A computer implemented method of evaluating the mental health of a person, said method comprising the steps of:
a. detecting and recording data from the person's autonomic and voluntary responses with one or more sensors in a first phase to obtain first phase data;
b. detecting and recording data from the person's autonomic and voluntary responses with one or more sensors during a second phase to obtain second phase data; and
c. comparing the first phase data with the second phase data to identify aberrations, deviations and/or patterns in the person's autonomic and voluntary responses,
wherein the aberrations, deviations and/or patterns are indicative of the presence or absence of one or more mental health ailments.

10. The method of claim 9, wherein historical data is compared with the first phase data and/or second phase data.

11. The method of claim 9, further comprising the step of evaluating the mental health of the person by a healthcare provider.

12. The method of claim 9, wherein the one or more sensors comprise a camera and a microphone.

13. The method of claim 9, wherein the one or more sensors comprise one or more of a camera, a microphone and at least one of a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor or a heart rate sensor.

14. The method of claim 9, further comprising the additional step of predicting whether the person will experience a mental health ailment.

15. The method of claim 9, further comprising the step of collecting data from the person with one or more sensors during one or more additional phases.

16. The method of claim 15, wherein data from each of the first phase, the second phase and the one or more additional phases is analyzed individually to identify aberrations, deviations and/or patterns that are indicative of the presence or absence of one or more mental health ailments.

17. The method of claim 15, wherein data from of each of the first phase, the second phase and the one or more additional phases is analyzed collectively to identify aberrations, deviations and/or patterns that are indicative of the presence or absence of one or more mental health ailments.

18. The method of claim 15, further comprising the step of predicting whether the person will experience a mental health ailment.

19. A computer implemented method of evaluating the mental health of a person, said method comprising the steps of:
a. collecting first phase data on the person's autonomic and voluntary responses with one or more sensors in a first phase;
b. detecting aberrations, deviations and/or patterns in the first phase data;
c. correlating the aberrations, deviations and/or patterns in the first phase data with one or more mental health ailments found in historical data;
d. collecting second phase data on the person's autonomic and voluntary responses with one or more sensors in a second phase;
e. detecting aberrations, deviations and/or patterns in the second phase data by comparing the second phase data with the first phase data; and
f. correlating the aberrations, deviations and/or patterns in the second phase data with one or more mental health ailments found in historical data,
wherein the aberrations, deviations and/or patterns detected in the first phase data and/or second phase data are indicative of the presence or absence of one or more mental health ailments.

20. The method of claim 19, wherein the one or more sensors comprise a camera and a microphone.

21. The method of claim 19, wherein the one or more sensors comprises one or more of a camera, a microphone and at least one of a respiration sensor, a handwriting sensor, an eye activity sensor, a pupilometer, a facial/micro-expression sensor, a body posture sensor, an accelerometer, a thermometer, a skin thermometer, a skin gas sensor, a skin conductivity sensor, a blood pressure sensor and a heart rate sensor.

22. The method of claim 19, further comprising the step of analyzing first phase data in conjunction with second phase data utilizing a fusion algorithm.

23. The method of claim 19, wherein the correlating steps of (c) and (f) utilize machine learning and/or artificial intelligence.

24. The method of claim 19, further comprising the step of predicting whether the person will experience a health ailment.

25. The method of claim 19, further comprising the steps of:
g. collecting data from the person's autonomic and voluntary responses with at least one sensor during one or more additional phases;
h. detecting aberrations, deviations and/or patterns in the data from the one or more additional phases by comparing the data from the one or more additional phases with the first phase data and second phase data; and
i. correlating the aberrations, deviations and/or patterns in the data from the one or more additional phases with one or more mental health ailments found in historical data.

26. The method of claim 25, wherein the detecting step (h) utilizes a fusion algorithm.

27. The method of claim 25, wherein the correlating step (i) utilizes machine learning and/or artificial intelligence.

28. The method of claim 25, further comprising the step of predicting whether the person will experience a mental health ailment.
